(19) United States
(12) Patent Application Publication
(10) Pub. No.: US 2005/0013829 A1
(43) Pub. Date: Jan. 20, 2005

(54) Novel growth factor Opa1 and uses thereof

(76) Inventor: David E. Weinstein, Dobbs Ferry, NY (US)

Correspondence Address: Leslie Gladstone Restaino;Brown Raysman Felder & Sleiner LLP, 163 Madison Avenue, P.O. Box 1989, Morristown, NJ, 07962-1989, US

(21) Appl. No.: 10/742,092

(22) Filed: Dec. 18, 2003

Related U.S. Application Data
(63) Continuation of application No. 09/479,145, filed on Jan. 7, 2000, now abandoned.
Application No. 09/479,145 is a continuation-in-part of application No. 09/294,764, filed on Apr. 19, 1999, now Pat. No. 6,569,423.

(51) Int. Cl. A61K 39/02; C07H 21/04; C07K 14/195

(52) U.S. Cl. 424/190.1; 435/69.3; 435/252.3; 435/320.1; 530/350; 536/23.7

(57) ABSTRACT

The present invention discloses nucleic acids encoding a novel growth factor Opa1, which promotes the regeneration of nervous tissue. Also disclosed are nucleic acids that hybridize to the complementary sequences of nucleic acids encoding Opa1, and vectors comprising said nucleic acids. In addition, the present invention also provides a purified Opa1 protein and methods of using the same.

### RELATED APPLICATION

This application is a continuation-in-part of co-pending U.S. Application No. 09/294,764, filed Apr. 19, 1999, the contents of which are expressly incorporated by reference herein.

### BACKGROUND OF THE INVENTION

Regeneration is a hallmark of the peripheral nervous system (PNS). This implies that following mechanical transection, regenerating PNS axons are capable of both finding their original target tissues and re-establishing functional synapses with a high degree of fidelity. In addition, the Schwann cells that were present before injury elaborate new myelin sheaths around the regenerated axons. The similarities between the developing and regenerating nerve, such as axon outgrowth and myelination, have led to the generally accepted view that regeneration recapitulates development. At some levels, this statement appears to be true: axons find their targets and glial cells recognize their cognate axons and regenerate their myelin organelles. However, in development, the axons serve as a template to guide Schwann cell migration, whereas in the regenerating nerve, axons migrate into a milieu in which the Schwann cells are pre-existing in situ. Moreover, the molecular events in the Schwann cell are different in the regenerating nerve as compared to the developing nerve.

Developmentally, peripheral axons reach their targets before most Schwann cells, the glia of the PNS, are born (Webster, 1984). Schwann cell progenitors proliferate and migrate along the axons, dividing the axon bundles into progressively smaller units, pinching off single axons, and establishing the Schwann cell-axon unit that is characteristic of the mature, myelinated nerve. However, following compression injury, peripheral nerves undergo a stereotyped pattern of Wallerian degeneration, characterized by myelin decompaction and autophagocytosis of the myelin debris, recruitment of cells of the monocyte/macrophage lineage and axonal die-back (reviewed in detail in Griffin et al., 1996). During the process of degeneration, the basal lamina, which had formally covered the myelinating Schwann cell/axon unit, is spared, thus leaving intact “endotubes” formed by the residual basal lamina and the associated Schwann cells which remain viable after axonal die-back. These endotubes form channels into which the regenerated axons will grow. Hence, in the regenerating nerve, axons extend toward their targets in the milieu of both the Schwann cell and the basal lamina, which is distinct from the milieu of the developing nervous system.

Following nerve injury, axons respond within hours by extending multiple new sprouts from the distal end of the proximal stump (Bray et al., 1972). One of these sprouts maneuvers into the pre-existing endotube, which will then grow toward the target, while the others are pruned away (Aguayo et al., 1973; Bray et al., 1972; McQuarrie, 1985). These observations have raised questions as to whether the endotube is necessary for axonal outgrowth, and if so, which component(s) of the endotube are active in promoting axon elongation. To establish the requirement for a basal lamina in nerve regeneration, several groups have created nerve transection models which leave a gap, or they have transected the nerve, leaving disjoined proximal and distal stumps. Under these conditions there is a failure of regeneration, associated with long term changes in gene expression in both the axon and the Schwann cell (Lisney, 1983; Meeker and Farel, 1993; Roytta and Salonen, 1988; Salonen et al., 1987; Watson et al., 1993; Weinberg and Spencer, 1978).

The basal lamina is a Schwann cell product (Bunge et al., 1982), the components of which, either alone or together, promote or permit axonal outgrowth when presented as a purified substratum (reviewed in Liuzzi and Tedeschi, 1991 and Luckenbill-Edds, 1997). These molecules include laminin (Combrooks et al., 1983), fibronectin (Tohyama and Ide, 1984), collagen type IV (Carey et al., 1983) and V, heparan sulfate proteoglycan (Mehta et al., 1985), tenascin (Zhang et al., 1995), and entactin (Baron-Van Evercooren et al., 1986). With the exception of a few positionally restricted molecules (e.g., s-laminin at the neuromuscular junction (Porter et al., 1995)), the basal lamina that coats the Schwann cell-axon unit is very similar to the other basal laminae found throughout the body (Martin et al., 1988; Yurchenco and Schittny, 1990). Taking advantage of this similarity, several types of basal laminae have been used as interpositional grafts into mixed sensory and motor nerves to promote axon regeneration. Various sources of material for engraftment have been tried, including acellular muscle basal lamina, acellular nerve basal lamina and acellular optic nerve. These grafts were made acellular by a variety of techniques, including repeated cycles of freeze-thaw, detergent lysis and hypotonic lysis (Feneley et al., 1991; Gulati, 1988; Hall, 1986; Hall and Kent, 1987; Ide et al., 1983; Sondell et al., 1998). While each of these paradigms showed some degree of anatomical and functional recovery, none showed the high degree of recovery observed if intact nerve, with viable Schwann cells in situ, was used as the bridging material (Hall, 1986). Interestingly, components of the basal lamina bind and present trophic factors to ingrowing axons (Albuquerque et al., 1998; Kagami et al., 1998; Rifkin et al., 1990), thereby raising questions about the combined sufficiency of the basal lamina and these factors in neural regeneration.

Electron microscopic examination of interpositional basal lamina grafts into myelinated peripheral nerve clearly demonstrated that axons enter the cell free grafts only when associated with co-migratory Schwann cells (Feneley et al., 1991). In a separate series of experiments, Enver and Hall used the anti-mitotic drug, mitomycin C, to block Schwann cell proliferation in rats treated with interpositional muscle basal lamina grafts in their peroneal nerves. In the absence of cell division, Schwann cells associated with the ingrowing axons, thereby demonstrating that the Schwann cells co-migrated with the axons rather than proliferating and migrating into unoccupied space within the graft (Enver and Hall, 1994). These studies showed a close approximation of the Schwann cell and axon, suggestive of an active, bi-directional communication in the penetration of the graft.

The inventor suggests that the co-migratory Schwann cell and axon be considered the “regenerating unit” when invading cell-free grafts. The absolute requirement for viable Schwann cells as part of the regenerating unit was demonstrated by preventing their migration into a gap created between the proximal and distal stumps of a transected nerve. Without the glial component of the regenerating unit there is a virtual block of axon outgrowth (Jenq and Coggeshall, 1987; Le Beau et al., 1988; Scaravilli et al., 1986).

The mechanism(s) employed by the Schwann cells to migrate in association with the ingrowing axons is not entirely known. However neuregulins in general, and the secreted form of neuregulin, GGF2, in particular (Marchionni et al., 1993), promote Schwann cell migration at doses below the Schwann cell mitotic threshold (Mahanthappa et al., 1996). The neuregulins are products of both motor and sensory axons (Chen et al., 1994; Marchionni et al., 1993; Meyer, 1995; Orr-Urtreger et al., 1993), thereby placing these molecules in close proximity to the Schwann cells in the ingrowing axon-Schwann cell units. In addition, high concentrations of GGF2 (500 ng/ml) induce Schwann cells to secrete a yet to be identified factor that promotes axonal branching and outgrowth (Mahanthappa et al., 1996). Very high local concentrations are possible in the microdomains of Schwann cell-axon contact. This elegant study suggests that the Schwann cell and the axon might in some manner be pulling and pushing one-another in the regenerating unit, and that bi-directional signaling is a critical aspect of the biology of regeneration.

The Schwann cell has a prodigious ability to synthesize a wide range of neurotrophic factors, including NGF, NT-3, BDNF, (Friedman et al., 1996; Lindsay, 1994; Maisonpierre et al., 1990), neuregulins (Rosenbaum et al., 1997), FGF1 and FGF2 (Fujimoto et al., 1997; Morgan et al., 1994; Neuberger and De Vries, 1993) and IGF1 and IGF2 (Hammarberg et al., 1998), ali of which are known to promote or support neurite outgrowth. These factors, alone or in combination, are incapable of replacing the Schwann cell in the regenerating unit. With the exception of FGF2, which showed early, but not sustained support of axon outgrowth in the absence of Schwann cells, there are no reports of trophic factors alone supporting complete or robust regeneration (Fujimoto et al., 1997). These observations raise the question as to the molecular basis of Schwann cell-mediated nerve regeneration.

Recent work by the inventor and colleagues elucidates the molecular genetic mechanisms employed by Schwann cells to regulate nervous tissue regeneration. During development, the transition from proliferating progenitor Schwann cells to myelinating Schwann cells is marked by a distinct developmental phase termed promyelination. During this phase the Schwann cells envest the axons, establishing the 1:1 relationship of the myelinating Schwann cell-axon unit (Webster, 1984). The histologic entry into promyelination is paralleled on the molecular level with the expression of the POU transcription factor SCIP (Weinstein et al., 1995), which is rapidly down-regulated at the onset of myelination (Monuki et al., 1990). During regeneration, SCIP is re-expressed as axons re-contact the resident Schwann cells. However, unlike development, SCIP expression is maintained in the Schwann cells after regeneration and remyelination (Scherer et al., 1994). The inventor and colleagues have recently reported on the accelerated and exaggerated regeneration of damaged peripheral nerves in transgenic animals expressing a mutant, activated form of SCIP (termed ΔSCIP) (Gondré et al., 1998). The transgene is expressed uniquely in the Schwann cells of the ΔSCIP animals (Weinstein et al., 1995). In addition to the overly exuberant regeneration observed in vivo, purified ΔSCIP Schwann cell populations support axonal out growth in vitro as well. These data show that Schwann cells are able to regulate the rate and extent of regeneration of the Schwann cell-axon unit in the damaged peripheral nerve. Attention is directed to the disclosure of co-pending application Ser. No. 09/294,764, filed Apr. 4, 1999, the complete contents of which are expressly incorporated herein by reference. In the instant case, the inventor reports on the isolation and identification of a novel growth factor from the ΔSCIP Schwann cells that induces nervous tissue regeneration, termed herein Opa1.

### SUMMARY OF THE INVENTION

The present invention is based upon the discovery of a novel protein and the nucleic acid encoding the same, said protein hereinafter denoted as Opa1. Studies by the inventor indicate that Opa1 is a growth factor involved in the regeneration of nervous tissue. This discovery may prove useful for the regeneration of nervous tissue in culture as well as for the treatment of subjects in need of nervous tissue regeneration, including subjects afflicted with neurodegenerative diseases or with otherwise damaged neurons. Such treatment may be effected by administration of Opa1 using a variety of delivery methods, including, but not limited to, gene therapy, including treatment with host cells or autologous cells transformed with a vector comprising a nucleic acid encoding Opa1, protein therapy or by the administration of agents which modulate Opa1 expression in an amount effective to induce or enhance expression of Opa1 and induce regeneration in the nervous tissues.

Accordingly, the present invention provides a purified and isolated nucleic acid encoding Opa1 protein, and more particularly, a purified and isolated nucleic acid comprising nucleotides 880-1680 of FIG. 2A or 2B. Also provided by the present invention is a nucleic acid probe which hybridizes to the complement of nucleic acid encoding Opa1, a mixture of nucleic acid probes each of which hybridizes to nucleic acid encoding Opa1 and a kit comprising one or more nucleic acid probes which hybridize to nucleic acid encoding Opa1. The present invention also provides a vector comprising a nucleic acid encoding Opa1 and a host cell transformed by this vector.

The present invention also provides a method for producing recombinant Opa1 comprising growing a bacterial or eukaryotic host cell transformed with a vector comprising nucleic acid encoding Opa1 in culture and recovering the recombinant Opa1 from the culture medium, from the host cell or from cell lysate. The present invention further provides a purified Opa1 protein, as well as an agent that binds to the Opa1 protein, including but not limited to an antibody immunoreactive with Opa1. In addition, the present invention provides a kit comprising an agent that binds to the Opa1 protein.

The present invention also provides a method for screening an agent that binds to a nucleic acid encoding Opa1 protein, comprising contacting the nucleic acid with an agent of interest and assessing the ability of the agent to bind to the nucleic acid. The present invention further provides a method for screening an agent that inhibits or promotes the expression of a nucleic acid encoding Opa1 protein, comprising the steps of contacting a cell transformed with a vector comprising the nucleic acid and assessing the effect of the agent on expression of the nucleic acid. The present invention still further provides a method for screening for an agent that binds to an Opa1 protein, comprising the steps of contacting the protein with an agent of interest and assessing the ability of the agent to bind to the protein.

Also provided is a method for evaluating the ability of an agent to induce nervous tissue regeneration, comprising the steps of contacting the candidate agent with nervous tissue and detecting the level of Opa1 expression in the nervous tissue, wherein an increased level of Opa1 expression may be indicative of nervous tissue regeneration.

The present invention also provides a recombinant viral vector capable of introducing nucleic acid encoding Opa1 into a target cell such that the target cell expresses Opa1, wherein the vector comprises (a) nucleic acid of or corresponding to at least a portion of the genome of a virus, the portion being capable of infecting the target cell, and (b) nucleic acid encoding a Opa1 protein operably linked to the viral nucleic acid.

The present invention further provides methods for regenerating nervous tissue comprising contacting the tissue with an effective amount of cells or host cells expressing Opa1 to regenerate the nervous tissue or, alternatively, comprising contacting the tissue with an effective amount of Opa1 to regenerate the nervous tissue.

Finally, the present invention also provides methods for treating a subject in need of nervous tissue regeneration, said methods comprising the administration to a subject of an effective amount of Opa1 to induce nervous tissue regeneration. The administration of Opa1 may be effected by a variety of methods, including, but not limited to, the introduction of an effective amount of cells expressing Opa1 into the subject to induce nervous tissue regeneration in the subject (including ΔSCIP Schwann cells or autologous cells transformed with a vector comprising a nucleic acid encoding Opa1), administration of the Opa1 protein, administration of the Opa1 nucleic acid, or by the administration of agents which modulate Opa1 expression in an amount effective to induce or enhance expression of Opa1.

Additional objects of the present invention will be apparent from the description which follows.

### BRIEF DESCRIPTION OF THE FIGURES

FIG. 1 depicts the results of two dimensional gel electrophoresis experiments, showing spots present in extracts from ΔSCIP Schwann cells that are not present in extracts from wild type Schwann cells.

FIG. 2 illustrates nucleic acid sequences encoding Opa1 from mouse (2A) and human (2B) cDNAs.

FIG. 3 depicts the results of Opa1 expression studies in various adult and developing mouse tissues.

FIG. 4 illustrates an analysis of Opa1 expression in the developing CNS, with three distinct bands that change in intensity over the course of development. Stages shown are E9.5, E13, P2 and P5. (E=embryonic; P=postnatal)

FIG. 5a shows that ΔSCIP cells constitutively express Opa1 mRNA, which is massively induced with 50 ng/ml of GGF. In contrast, the wild type Schwann cells express no Opa1 at baseline and are induced to the baseline ΔSCIP level with the addition of 50 ng/ml GGF.

FIG. 5b is a dose response curve of GGF on wild type Schwann cells, which shows that expression of Opa1 plateaus at 50 ng/ml GGF.

FIG. 6 illustrates modulation of Opa1 expression in cultured Schwann cells in the presence of the neuroimmunophilin FK506. A dose curve of FK506, dissolved in DMSO, was added to primary mouse Schwann cells and assayed for Opa1 induction after 48 hours. Strong induction is seen at 10 nM and maximal induction at 100 nM, although above this dose, Opa1 expression precipitously falls. The induction of Opa1 with FK506 corresponds to the reported dosages at which FK506 has a therapeutic effect on nerve regeneration in animal models.

FIG. 7a depicts the outgrowth of axons of PC12 cells cultured on wild type and Opa1 expressing cells. Non-differentiated PC12 cells cultured on Opa1 expressing Schwann cells extend axons that approach 100 μm overnight.

FIG. 7b depicts granule cell survival and axon outgrowth of cerebellar granule cells cultured on monolayers of wild type and Opa1 expressing Schwann cells. There was a tremendous difference in granule cell survival in the presence of Opa1, as well as tremendous axonal outgrowth.

FIG. 8 depicts cell survival (Panel A) and axon outgrowth (Panel B) of cerebellar granule cells cultured on monolayers of Opa1 expressing Schwann cells.

### DETAILED DESCRIPTION OF THE INVENTION

As used herein, the following words and phrases have the meaning set forth below:

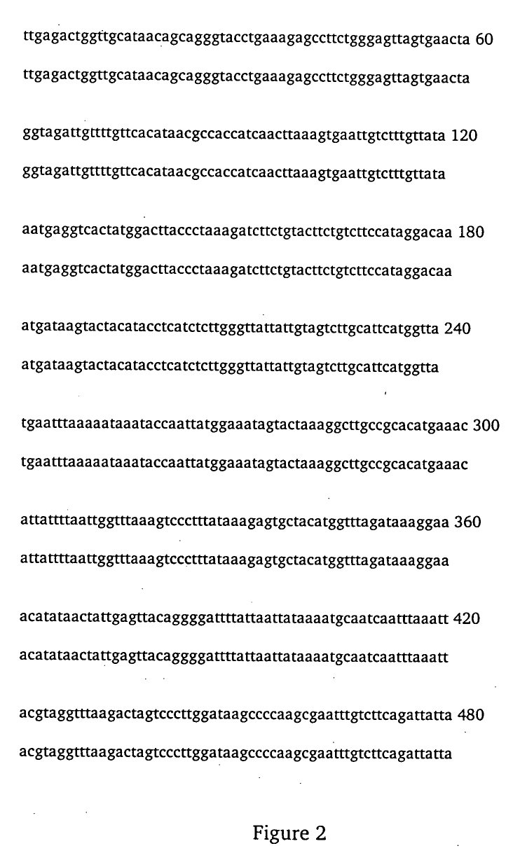

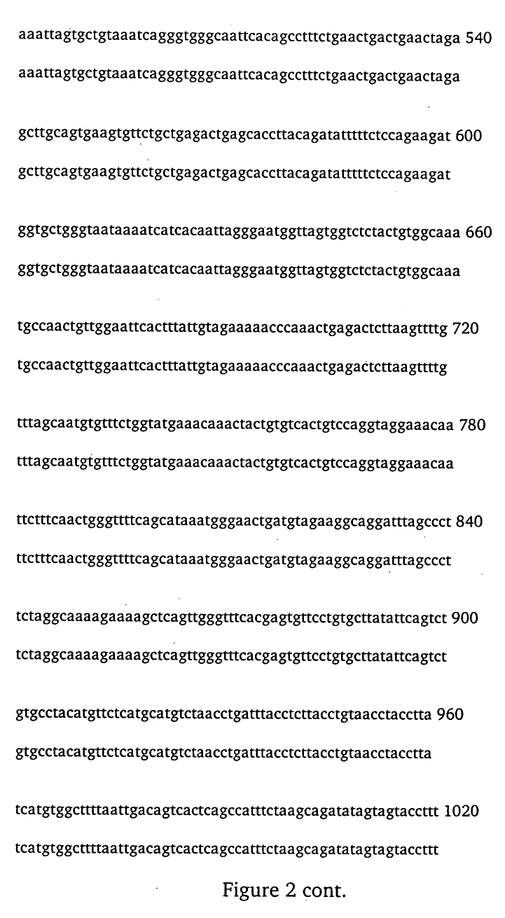

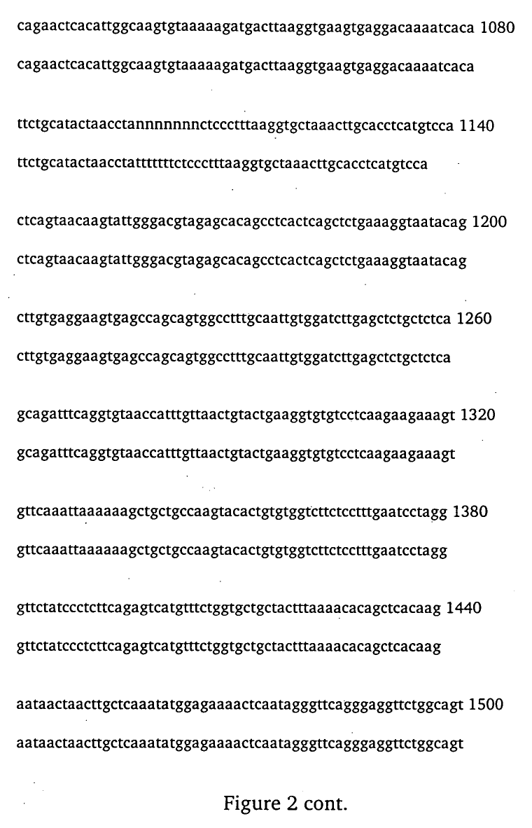

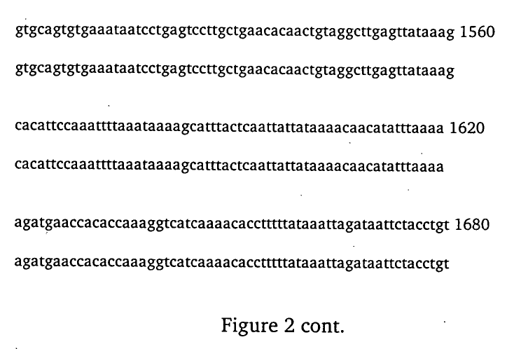

“Opa1 protein” includes, where appropriate, both the Opa1 protein and analogues of Opa1 protein, wherein an analogue of Opa1 may be any protein having functional similarity to the Opa1 protein, and which (a) also possesses certain regions that are conserved among the Opa1 family members or (b) is encoded by a nucleic acid comprising a nucleotide sequence that is at least 80%, 85%, 90%, 95%, or 98% identical to nucleotides 880-1680 of FIG. 2A or 2B.

The term “nervous tissue” as used herein includes nervous tissue present in both the central nervous system and the peripheral nervous system, and most generally includes, but is not limited to, neurons and neuroglia, and discrete parts of neurons and neuroglia. “Neurons” are any of the conducting or nerve cells of the nervous system that typically consist of a cell body containing the nucleus and surrounding cytoplasm (perikaryon), several short radiating processes (dendrites), and one long process (the axon), which terminates in twiglike branches (telodendrons) and may have branches (collaterals) projecting along its course. “Neuroglia” are the neuroglial cells or glial cells which form the supporting structure of the nervous tissue. Nervous tissue is also meant to encompass discrete parts of the neurons and neuroglia.

Non-exclusive specific examples of various types of nervous tissue include, but are not limited to, any or all of the following: Schwann cells, stellate cells, satellite cells, astrocytes, oligodendrocytes, any type of granular cell, cells contained in the ganglia, grey matter, or white matter, myelin, neurilemma, axons, dendrites, motor neurons, fibrils and fibular processes.

The terms “Opa1 biological activity”, “Opa 1 function” or “neuroregenerative biological activity” are meant to refer to functions normally performed by wild-type Opa1 (or an analogue of Opa1). Such functions can include axonogenesis of a nerve, the myelination of a nerve, the growth of neurons, the growth of neuroglia, the growth of the axons or dendrites of a nerve, the growth of fibrils of neuroglia, the growth of stellate cells, the growth of fibular processes of neuroglia, the remyelination of grey matter, and the remyelination of white matter. The neuroregenerative biological activity may take place in nerves of both the central nervous system and the peripheral nervous system.

As used herein, “growth” may be defined as an increase in thickness, diameter, and length of the nerve fibers or the myelin or neurilemma coverings, and the supporting fibrils and fibular processes. The definition of “growth” as used herein also includes an increase in the numbers of Schwann cells, stellate cells or neuroglial cells present on or supporting a nerve.

Unless otherwise indicated, “protein” shall mean a protein, polypeptide or peptide.

The present invention provides a purified and isolated nucleic acid encoding an Opa1 protein. As used herein, the nucleic add may be genomic DNA, cDNA, RNA or antisense RNA and includes nucleic acid derived from any species, and preferably from a mammalian species, e.g., a human or a mouse. Due to the degeneracy of the genetic code, the nucleic acid of the present invention also includes a multitude of nucleic acid substitutions which will also encode Opa1. The nucleic acid from the mouse preferably comprises the nucleotide sequence as shown in FIG. 2A and more preferably comprises nucleotides 880-1680 of FIG. 2A. The nucleic acid from a human preferably comprises the nucleotide sequence shown in FIG. 2B, and more preferably comprises nucleotides 880-1680 of FIG. 2B.

In addition, the present invention provides the nucleic acid encoding Opa1 protein having one or more mutations resulting in the expression of either a non-functional or mutant protein, or in lack of expression altogether. The mutation may be one or more point, insertion, rearrangement or deletion mutations or a combination thereof.

The present invention also includes an isolated and purified nucleic acid comprising the nucleic acid of FIG. 2A or 2B or a contiguous fragment thereof (said fragment comprising most preferably nucleotides 880 to 1680 of FIG. 2A or 2B, respectively), wherein said nucleic acid encodes a protein having Opa1 biological activity. Also disclosed is an isolated nucleic acid that hybridizes under high stringency conditions (i.e., hybridization to filter bound DNA in 0.5M NaHPO₄ at 65° C. and washing in 0.1×SSC/0.1% SDS at 68° C.) or moderate stringency conditions (i.e., washing in 0.2×SSC/0.1% SDS at 42° C.) (Ausubel, F. M. et al., 1998 Current Protocols in Molecular Biology) to the complement of the nucleic acid sequence of FIG. 2A or 2B, or to a contiguous fragment thereof (said fragment comprising most preferably nucleotides 880 to 1680 of FIG. 2A or 2B, respectively), wherein said isolated nucleic acid encodes a protein having Opa1 biological activity.

Also disclosed are isolated nucleic acids that encode a protein having Opa1 biological activity, wherein said nucleic acids comprise a nucleic acid sequence that is at least 80%, preferably at least 85%, more preferably at least 90%, even more preferably at least 95%, and most preferably at least 98%, identical to the nucleic acid sequence of FIG. 2A or 2B, or to a contiguous fragment of FIG. 2A or 2B (said fragment comprising most preferably nucleotides 880 to 1680 of FIG. 2A or 2B, respectively).

The present invention also provides an isolated and substantially purified Opa1 protein or Opa1 analogue and includes Opa1 isolated from tissue obtained from a subject or recombinantly produced as described below. Preferably, the Opa1 protein or Opa1 analogue of the present invention is encoded by the nucleic acid of FIG. 2A or 2B, or a contiguous fragment thereof (said fragment comprising most preferably nucleotides 880-1680 of FIG. 2A or 2B, respectively), and has a molecular weight of around 100 kDa. Alternatively, the Opa1 protein or Opa1 analogue of the present invention is encoded by a nucleic acid that hybridizes to a complement of the nucleic acid of FIG. 2A or 2B (or to a contiguous fragment thereof, said fragment comprising most preferably nucleotides 880-1680 of FIG. 2A or 2B, respectively) under high stringency or moderate stringency conditions, wherein said nucleic acid encodes a protein having Opa1 biological activity. As used herein, an “analogue” may be any protein having functional similarity to the Opa1 protein, that also possesses certain regions that are conserved among the Opa1 family members or is encoded by a nucleic acid comprising a nucleotide sequence that is at least 80%, 85%, 90%, 95%, or 98% identical to nucleotides 880-1680 of FIG. 2A or 2B. The present invention also includes a non-functional Opa1 protein, i.e., Opa1 which is inactive or only has minimal effects in vivo or in vitro. The non-functional Opa1 protein may have one or more deletions or substitutions of its amino acid sequence that results in the Opa1 protein losing its functionality.

The present invention also provides a vector which comprises a nucleic acid encoding an Opa1 protein or an Opa1 analogue, including a nucleic acid which hybridizes under high stringency conditions or moderate stringency conditions to the complement of the nucleic acid sequence of FIG. 2A or 2B, or to a contiguous fragment thereof (said fragment comprising most preferably nucleotides 880 to 1680 of FIG. 2A or 2B, respectively). Such vectors may be constructed by inserting nucleic acid encoding Opa1 or an Opa1 analogue into a suitable vector nucleic acid, operably linked to an expression control sequence as described below. The term “inserted” as used herein means the ligation of a foreign DNA fragment and vector DNA by techniques such as the annealing of compatible cohesive ends generated by restriction endonuclease digestion or by use of blunt end ligation techniques. Other methods of ligating DNA molecules will be apparent to one skilled in the art.

The vectors of the present invention may be derived from a number of different sources, including plasmids, viral-derived nucleic acids, lytic bacteriophage derived from phage lambda, cosmids or filamentous single-stranded bacteriophages such as M13. Depending upon the type of host cell into which the vector is introduced, vectors may be bacterial or eukaryotic. Bacterial vectors are derived from many sources including the genomes of plasmids and phages. Eukaryotic vectors are also constructed from a number of different sources, e.g. yeast plasmids and viruses. Some vectors, called shuttle vectors, are capable of replicating in both bacteria and eukaryotes. The nucleic acid from which the vector is derived is usually greatly reduced in size so that only those genes essential for its autonomous replication remain. The reduction in size enables the vectors to accommodate large segments of foreign DNA.

Examples of suitable vectors into which the nucleic acid encoding the Opa1 protein can be inserted include but are not limited to pCGS, pBR322, pUC18, pUC19, pHSV-106, pJS97, pJS98, M13 mp18, M13 mp19, pSPORT 1, pGem, pSPORT 2, pSV SPORT 1, pBluescript II, λZapII, λgt10, λgt11, λgt22A, and λZIPLOX. Other suitable vectors are obvious to one skilled in the art.

The vector of the present invention may be introduced into a host cell and may exist in integrated or unintegrated form within the host cell. When in unintegrated form, the vector is capable of autonomous replication. The term “host cell” as used herein means the bacterial or eukaryotic cell into which the vector is introduced. As used herein, “introduced” is a general term indicating that one of a variety of means has been used to allow the vector to enter the intracellular environment of the host cell in such a way that it exists in stable and expressable form therein.

Some bacterial and eukaryotic vectors have been engineered so that they are capable of expressing inserted nucleic acids to high levels within the host cell. An “expression cassette” or “expression control sequence” comprising nucleic acid encoding an Opa1 protein operably linked or under the control of transcriptional and translational regulatory elements (e.g. a promoter, ribosome binding site, operator, or enhancer) can be made and used for expression of Opa1 protein in vitro or in vivo. The choice of regulatory elements employed may vary, depending for example on the host cell to be transfected and the desired level of expression. For example, in vectors for the expression of a gene in a bacterial host cell such as E. coli, the lac operator-promoter or the tac promoter are often used. Eukaryotic vectors use promoter-enhancer sequences of viral genes, especially those of tumor viruses. Several promoters for use in mammalian cells are known in the art and include, for example, the phosphoglycerate (PGK) promoter, the simian virus 40 (SV40) early promoter, the Rous sarcoma virus (RSV) promoter, the adenovirus major late promoter (MLP) and the human cytomegalovirus (CMV) immediate early 1 promoter. However, any promoter that facilitates suitable expression levels can be used in the instant invention. Inducible promoters, (e.g. those obtained from the heat shock gene, metallothionein gene, beta interferon gene, or steroid hormone responsive genes, including but not limited to the lac operator-promoter in E. coli or metallothionine or mouse mammary tumor virus promoters in eukaryotic cells) may be useful for regulating transcription based on external stimuli. As used herein, “expression” refers to the ability of the vector to transcribe the inserted nucleic acid into mRNA so that synthesis of the protein encoded by the inserted nucleic acid can occur.

Vectors suitable for the expression of the nucleic acid encoding Opa1 in a host cell are well known to one skilled in the art and include pET-3d (Novagen), pProEx-1 (Life Technologies), pFastBac 1 (Life Technologies), pSFV (Life Technologies), pcDNA II (Invitrogen), pSL301 (Invitrogen), pSE280 (Invitrogen), pSE380 (Invitrogen), pSE420 (Invitrogen), pTrcHis. A,B,C (Invitrogen), pRSET A,B,C (Invitrogen), pYES2 (Invitrogen), pAC360 (Invitrogen), pVL1392 and pV11392 (Invitrogen), pCDM8 (Invitrogen), pcDNA I (Invitrogen), pcDNA I(amp) (Invitrogen), pZeoSV (Invitrogen), pcDNA 3 (Invitrogen), pRc/CMV (Invitrogen), pRc/RSV (Invitrogen), pREP4 (Invitrogen), pREP7 (Invitrogen), pREP8 (Invitrogen), pREP9 (Invitrogen), pREP10 (Invitrogen), pCEP4 (Invitrogen), pEBVHis (Invitrogen), and λPop6. Other vectors would be apparent to one skilled in the art.

Vectors may be introduced into host cells by a number of techniques known to those skilled in the art, e.g. via calcium phosphate or calcium chloride co-precipitation, DEAE dextran mediated transfection, lipofection, electroporation, cationic liposome fusion, protoplast fusion, DNA coated-microprojectile bombardment, and infection with recombinant replication-defective retroviruses. The term “transformation” denotes the introduction of a vector into a bacterial or eukaryotic host cell. As such, it encompasses transformation of bacterial cells and transfection, transduction and related methods in eukaryotic cells.

Any one of a number of suitable bacterial or eukaryotic host cells may be transformed with the vector of the present invention, including various neuroglia, such as Schwann cells. Examples of suitable host cells are known to one skilled in the art and include but are not limited to bacterial cells such as E. coli strains c600, c600hf1, HB101, LE392, Y1090, JM103, JM109, JM101, JM107, Y1088, Y1089, Y1090, Y1090(ZZ), DM1, PH10B, DH11S, DH125, RR1, TB1 and SURE, Bacillus subtilis Agrobacterium tumefaciens, Bacillus megaterium; and eukaryotic cells such as Pichia pastoris, Chlamydomonas reinhardtii, Cryptococcus neoformans, Neurospora crassa, Podospora anserina, Saccharomyces cerevisiae, Saccharomyces pombe, Uncinula necator, cultured insect cells, cultured chicken fibroblasts, cultured hamster cells, cultured human cells such as HT1080, MCF7, and 143B, and cultured mouse cells such as EL4 and NIH3T3 cells.

The present invention also provides a method for producing a recombinant Opa1 protein comprising introducing a nucleic acid encoding Opa1 (or a nucleic acid that hybridizes under high stringency conditions or moderate stringency conditions to the complement of a nucleic acid encoding Opa1 or a contiguous fragment thereof, where said nucleic acid encodes a protein with Opa1 biological activity) into a suitable bacterial or eukaryotic host cell (including a neuroglial cell, such as a Schwann cell), maintaining said host cells under conditions whereby the nucleic acid is expressed to produce Opa1, and recovering recombinant Opa1 from the culture medium, from the host cells or from cell lysate. As used herein the term “recombinant” refers to Opa1 produced by purification from a host cell transformed with a vector capable of directing its expression to a high level.

A variety of methods of growing host cells transformed with a vector are known to those skilled in the art. The type of host cell, i.e., bacterial or eukaryote, is the primary determinant of the method to be utilized and the optimization of specific parameters relating to such factors as temperature, trace nutrients, humidity, and growth time. Depending on the vector, the host cells may have to be induced by the addition of a specific compound at a certain point in their growth cycle in order to initiate expression of the nucleic acid of the present invention. Examples of compounds used to induce expression of the nucleic acid of the present invention are known to one skilled in the art and include but are not limited to IPTG, zinc and dexamethasone. Using standard methods of protein isolation and purification, such as ammonium sulfate precipitation followed by dialysis to remove salt, followed by fractionation according to size, charge of the protein at specific pH values, affinity methods, etc., recombinant Opa1 may be extracted from suitable host cells transformed with vector capable of expressing the nucleic acid encoding Opa1.

The present invention also provides for agents that bind to the Opa1 protein and analogues thereof, as well as the non-functional Opa1 protein. The agent may be a antibody, a nucleic acid, a protein, a peptide, DNA, RNA, mRNA, antisense RNA, a drug or a compound. Agents that bind to the Opa1 protein or an analogue thereof may be identified or screened by contacting the protein with the agent of interest and assessing the ability of the agent to bind to the protein.

Antibodies immunoreactive with Opa1 or analogues thereof include antibodies immunoreactive with non-functional Opa1 protein. The antibodies of the present invention may be monoclonal or polyclonal and are produced by techniques well known to those skilled in the art, e.g., polyclonal antibody can be produced by immunizing a rabbit, mouse, or rat with purified Opa1 and monoclonal antibody may be produced by removing the spleen from the immunized rabbit, mouse or rat and fusing the spleen cells with myeloma cells to form a hybridoma which, when grown in culture, will produce a monoclonal antibody. Labeling of the antibodies of the present invention may be accomplished by standard techniques using one of the variety of different chemiluminescent and radioactive labels known in the art. The antibodies of the present invention may also be incorporated into kits which include an appropriate labeling system, buffers and other necessary reagents for use in a variety of detection and diagnostic applications.

The present invention provides for agents that bind to a nucleic acid encoding Opa1 protein. Suitable agents include but are not limited to a nucleic acid, a protein, a peptide, DNA, RNA, mRNA, antisense RNA, a drug or a compound. The agents may inhibit or promote expression of the Opa1 nucleic acid. Such agents may be discovered by a method for screening for an agent that binds to the nucleic acid of Opa1 comprising contacting the nucleic acid with an agent of interest and assessing the ability of the agent to bind to the nucleic acid. An agent that inhibits or promotes the expression of the nucleic acid encoding the Opa1 protein may be screened by contacting a cell transformed with a vector comprising the nucleic acid, and assessing the effect of the agent on expression of the nucleic acid.

The present invention also provides nucleic acid probes and mixtures thereof which are hybridizable to the nucleic acid encoding the Opa1 protein. Such probes may be prepared by a variety of techniques known to those skilled in the art such as PCR and restriction enzyme digestion of Opa1 nucleic acid or by automated synthesis of oligonucleotides whose sequence correspond to selected portions of the nucleotide sequence of the Opa1 nucleic acid using commercially available oligonucleotide synthesizers such as the Applied Biosystems Model 392 DNA/RNA synthesizer. The nucleic acid probes of the present invention may also be prepared so that they contain one or more point, insertion, rearrangement or deletion mutations or a combination thereof to correspond to mutations of the Opa1 gene. The nucleic acid probes of the present invention may be DNA or RNA and may vary in length from about 8 nucleotides to the entire length of the Opa1 nucleic acid. Preferably, the probes are 8 to 30 nucleotides in length, and even more preferably correspond to nucleotides within bp880-1680 of FIG. 2A or 2B. Labeling of the nucleic acid probes may be accomplished using one of a number of methods known in the art, e.g., PCR, nick translation, end labeling, fill-in end labeling, polynucloetide kinase exchange reaction, random priming, or SP6 polymerase (for riboprobe preparation) and one of a variety of labels, e.g., radioactive labels such as ³⁵S, ³²p or ³H or nonradioactive labels such as biotin, fluorescein (FITC), acridine, cholesterol or carboxy-X-rhodamine (ROX). Combinations of two or more nucleic probes corresponding to different or overlapping regions of the Opa1 nucleic acid may also be included in kits for use in a variety of detection and diagnostic applications.

The present invention also provides a method for evaluating neuroregenerative biological activity associated with heightened Opa1 expression in a subject's cells. Neuroregenerative activity may be evaluated in a patient, including a patient with neurodegenerative disease such as Alzheimer's, Pick's disease, Huntington's disease, Parkinson's disease, cerebral palsy, amyotrophic lateral sclerosis, muscular dystrophy, multiple sclerosis, myasthena gravis or Binswanger's disease, by detecting increased or decreased expression of Opa1 using nucleic acid hybridization and/or immunological techniques well known in the art.

For example, nucleic acid hybridization using mRNA extracted from cells and Opa1 nucleic acid probes can be used to determine the concentration of Opa1 mRNA present in the cell and the concentration thus obtained compared to the value obtained for cells which exhibit a normal level of Opa1 activity. Isolation of RNA from cells is well known in the art and may be accomplished by a number of techniques, e.g., whole cell RNA can be extracted using guanidine thiocyanate; cytoplasmic RNA may be prepared by using phenol extraction methods; and polyadenylated RNA may be selected using oligo-dT cellulose. Alternatively, the concentration of Opa1 may be determined from binding studies using labeled antibody immunoreactive with Opa1.

Further, the present invention provides a method for evaluating agents that may induce nervous tissue growth or regeneration, comprising contacting the candidate agent with nervous tissue, and detecting the level of Opa1 expressed using nucleic acid hybridization and/or immunological techniques known in the art and as described above, wherein an increased level of Opa1 expression may be indicative of growth or regeneration in the nervous tissue. In this manner, agents may be screened for their neuroregenerative activity using Opa1 as an indicator that nervous tissue growth or regeneration has recently occurred or is occurring. Similarly, such methods may be used to evaluate the efficacy of an agent or therapeutic administered to a subject in need of nervous tissue regeneration by simply measuring the level of Opa1 expression in the nervous tissue of said patient, wherein a heightened level of Opa1 expression may be indicative of nervous tissue growth or regeneration.

Neurological defects resulting from mutations in the nucleic acid encoding Opa1 may be detected by one of a number of methods known in the art, e.g., hybridization analysis of nucleic acid extracted from a sample of tissue or cells from a subject using nucleic acid probes designed to detect the presence of mutations in the nucleic acid encoding Opa1. Alternatively, the defect may be detected using antibody immunoreactive with non-functional Opa1 and standard immunological detection techniques such as Western blotting.

The present invention also provides a method of inducing nervous tissue growth or regeneration comprising administering to said tissue an effective amount of Opa1 or an Opa1 analogue to regenerate the nervous tissue. The administration of Opa1 or Opa1 analogue may be effected by a variety of methods, including, but not limited to, contacting the nervous tissue with an effective amount of cells expressing Opa1 (including ΔSCIP Schwann cells, or host cells transformed with a vector comprising nucleic acid encoding Opa1), administration of the Opa1 protein, administration of the Opa1 nucleic acid, or by the administration of agents (such as GGF or FK506) which modulate Opa1 expression in an amount effective to induce or enhance expression of Opa1. It is understood that nervous tissue growth or regeneration, as used herein, includes axonogenesis of a nerve, the myelination of a nerve, the growth of neurons, the growth of the axons or dendrites of a nerve, the growth of fibrils of neuroglia, the growth of stellate cells, the growth of fibular processes of neuroglia, the remyelination of grey matter, and the remyelination of white matter.

Also provided by the present invention is a method of inducing nervous tissue growth or regeneration in a subject in need of nervous tissue growth or regeneration comprising administering to the subject an effective amount of Opa1 or an Opa1 analogue to induce nervous tissue growth or regeneration in the subject. The neuroregenerative activity of Opa1 renders Opa1 useful for preventing the onset or reducing the severity of damaged or degenerated nervous tissue. The subject in need of nervous tissue growth or regeneration may have a neurodegenerative disease, damaged neurons and/or damaged myelin. Non-limiting examples of such neurodegenerative diseases are Alzheimer's disease, Pick's disease, Huntington's disease, Parkinson's disease, cerebral palsy, amyotrophic lateral sclerosis, muscular dystrophy, multiple sclerosis, myasthenia gravis, and Binswanger's disease. In addition, damaged neurons or myelin caused by vascular lesions of the brain and spinal cord, trauma to the brain and spinal cord, cerebral hemorrhage, intracranial aneurysms, hypertensive encephalopathy, subarachanoid hemorrhage or developmental disorders may also be treated using the methods provided by the present invention. Examples of developmental disorders include, but are not limited to, a defect of the brain, such as congenital hydrocephalus, or a defect of the spinal cord, such as spina bifida.

The administration of Opa1 or the Opa1 analogue may be effected by a number of different routes, including administration of the Opa1 or Opa1 analogue protein itself, administration of a nucleic acid encoding Opa1 or Opa1 analogue by the use of standard DNA techniques, by treatment with an effective amount of cells expressing Opa1 (including ΔSCIP Schwann cells, or host cells transformed with a vector comprising nucleic acid encoding Opa1), treatment with autologous cells (preferably neuroglial cells, such as Schwann cells) transformed with nucleic acid encoding Opa1, or by administration of agents (such as GGF or FK506) which modulate Opa1 expression in an amount effective to induce or enhance expression of Opa1.

As a part of protein therapy, the Opa1/Opa1 analogue may be administered to a tissue or subject in conjunction with a pharmaceutically acceptable carrier or diluent topically on an exposed nerve, nervous tissue, or transplant tissue, or by intravenous, intramuscular, intradermal, subcutaneous or intraperitoneal injection or other appropriate route of administration in an effective dosage range. The Opa1/Opa1 analogue is administered in amounts sufficient to promote nervous tissue regeneration in a subject, and may be administered alone or in association with an agent that facilitates passage (i.e., via fusion or endocytosis) through cell membranes or that promotes Opa1 biological activity. Opa1 or Opa1 analogue may be produced synthetically or recombinantly, or may be isolated from native cells.

A nucleic acid encoding Opa1 or Opa1 analogue may also be administered to a subject using gene therapy, i.e. by the administration of a recombinant vector containing a nucleic acid sequence encoding the Opa1 or Opa1 analogue protein. The nucleic acid sequence encoding Opa1/Opa1 analogue administered to a subject may be genomic DNA or cDNA. The nucleic acid sequence may be administered using a number of procedures known to one skilled in the art, such as electroporation, DEAE Dextran, monocationic liposome fusion, polycationic liposome fusion, protoplast fusion, DNA coated microprojectile bombardment, by creation of an in vivo electrical field, injection with recombinant replication-defective viruses (including retroviruses and adeno-associated viruses), homologous recombination, receptor mediated entry and naked DNA transfer. It is to be appreciated by one skilled in the art that any of the above methods of DNA transfer may be combined.

The recombinant vector may comprise a nucleic acid of or corresponding to at least a portion of the genome of a virus, where this portion is capable of directing the expression of a nucleic sequence encoding Opa1 protein, operably linked to the viral nucleic acid and capable of being expressed as a functional gene product in the subject.

The recombinant vectors may also contain a nucleotide sequence encoding suitable regulatory elements so as to effect expression of the vector construct in a suitable host cell. As used herein, “expression” refers to the ability of the vector to transcribe the inserted DNA sequence into mRNA so that synthesis of the protein encoded by the inserted nucleic acid can occur. Those skilled in the art will appreciate that a variety of enhancers and promoters are suitable for use in the constructs of the invention, and that the constructs will contain the necessary start, termination, and control sequences for proper transcription and processing of the nucleic acid sequence encoding Opa1 protein when the recombinant vector construct is introduced into a mammal. Vectors suitable for the expression of the nucleic sequence encoding Opa1 protein are well known to one skilled in the art.

For the purposes of gene transfer into a cell, tissue or subject, a recombinant vector containing nucleic acid encoding Opa1 may be combined with a sterile aqueous solution which is preferably isotonic with the blood of the recipient. Such formulations may be prepared by suspending the recombinant vector in water containing physiologically compatible substances such as sodium chloride, glycine, and the like, and having buffered pH compatible with physiological conditions to produce an aqueous solution, and rendering such solution sterile. In a preferred embodiment of the invention, the recombinant vector is combined with a 20-25% sucrose in saline solution in preparation for introduction into a mammal.

The amounts of nucleic acid encoding Opa1, or nucleic acid encoding Opa1 contained in a vector are administered in amounts sufficient to induce nervous tissue regeneration in a subject. However, the exact dosage will depend on such factors as the purpose of administration, the mode of administration, and the efficacy of the composition, as well as the individual pharmacokinetic parameters of the subject. Such therapies may be administered as often as necessary and for the period of time as judged necessary by one of skill in the art.

The invention further provides that Opa1 may be administered to a subject in need of nervous tissue regeneration by introducing an effective amount of neuroglial cells (such as Schwann cells or ΔSCIP Schwann cells) or other host cells expressing Opa1 or an Opa1 analogue into the subject to induce nervous tissue regeneration in the subject.

In an alternative embodiment of the invention, biologically active Opa1 may be provided to the cell of an individual in need of nervous tissue growth or regeneration comprising isolating autologous host cells (preferably neuroglial cells, and most preferably, Schwann cells) from the individual, transforming the isolated host cells with an expression vector that contains and expresses the nucleic acid encoding Opa1, and transplanting the autologous host cells into the individual in need of nervous tissue regeneration so as to provide biologically active Opa1 to a cell or tissue of said individual. The vector may be any vector as described above, but is most preferably a recombinant replication defective virus, including, but not limited to, retroviruses or adeno-associated viruses. The transformed host cells may be assayed for transduction efficiency by techniques known to those skilled in the art, including PCR identification of the Opa1 nucleic acid, Southern blot determination of gene copy number, enzymatic assay, hybridization with nucleic acid probes, and immunocytochemistry to detect the Opa1 protein. Upon the establishment of an autologous transformed host cell population, the cells are reintroduced back into the subject in need of nervous tissue growth or regeneration.

The invention further provides a method for inducing nervous tissue growth or regeneration in a subject comprising contacting the nervous tissue of said subject with a modulator of Opa1 expression in an amount effective to induce or enhance expression of Opa1 and induce growth or regeneration in the nervous tissues of said subject. The modulator may be a protein, nucleic acid, compound, or agent that induces Opa1 expression, including but not limited to GGF or FK506.

It is within the confines of the invention that Opa1 may be administered in combination with one or more growth and/or regulatory factors to promote nervous tissue regeneration. Opa1 may be administered to a subject prior to, simultaneously with or subsequent to administration of a growth factor and/or regulatory factor.

Finally, since Opa1 induces nervous tissue growth or regeneration, Opa1 may be useful for enhancing wound healing, organ regeneration, organ transplantation (e.g., heart, kidney, lung, and liver), the transplantation of artificial organs, and in the acceptance of grafts (e.g skin, appendages, etc.).

The present invention may be better understood by reference to the following non-limiting Example. The following Example is presented in order to more fully illustrate the preferred embodiments of the invention, and should in no way be construed as limiting the scope of the present invention.

### EXAMPLE

Materials and Methods

Cell Culture for Outgrowth Assay

A. Schwann cells: On the day before establishment of co-cultures, 30 000 of either wild-type, primary mouse or ΔSCIP primary Schwann cells were added per well of a 24 well plate and cultured in D¹⁰ (DMEM supplemented with 10% of FCS, 1× non essential amino acids, penicillin, streptomycin, glutamine and 250 ng/ml Fungizone) over night, as described (Wu and Weinstein, 1999). PC 12 Cells were grown in DMEM supplemented with 10% FCS and 5% horse serum, 1× nonessential amino acids, penicillin, streptomycin, glutamine and 250 ng/ml Fungizone. The cells were grown on tissue culture precoated with 2% rat tail collagen, as described (Weinstein et al., 1990). Cerebellar granular cells from P2 rats were prepared and partially purified on a Percoll gradient. The contaminating astrocytes were removed by differential adhesion to tissue culture plastic, all as described (Weinstein, 1997).

B. Co-cultures: The Schwann cell culture medium was removed and 10⁴ of either PC12 cells or cerebellar granular cells were added in DMEM supplemented with 10% FCS and 5% horse serum, 1× nonessential amino acids, penicillin, streptomycin, glutamine and 250 ng/ml Fungizone, and cultured overnight.

Immunostaining

Cultures were rinsed with PBS and fixed with PBS buffered 4% paraformaldehyde, pH 7.5. The cells were permeabilized and blocked in 10% normal goat serum/0.1% Triton X100 for 1 h at room temperature. The anti neuron-specific tubulin antibody TuJ 1 was added 1:1000 for 1 h at room temperature, then washed five times in PBS/0.01% Triton X100 and then exposed to goat anti-mouse/biotin for 1 h at room temperature. The cells were washed five times in PBS/0.01% Triton X-100-100 and the secondary antibody was visualized using the ABC elite system (Vector Laboratory) with DAB and nickel. The stained co-cultures were photographed, and scanned using Adobe Photoshop. The images were subsequently analyzed with the MetaMorph imaging system in the manual mode. In brief, each neurite was marked and its course was marked by tracing with the mouse. The same procedure was carried out for all for secondary neurites. The outline of each process was then converted to pixels, the results were then transferred to a Microsoft Excel spreadsheet. Statistical evaluations were made with the statistical function of Excel, using a student unpaired T-test with unequal variances.

Cell Culture for 2D Electrophoresis

Wild-type mouse Schwann cells and ΔSCIP Schwann cells were cultured for 24 hours in serum-free conditions in a medium supplemented with insulin (5 mg/L) transferin (10 mg/L) and selenium (sodium selenite 30 nM), after which the medium was aspirated and the cells were rinsed thrice with PBS. The cells were then scraped into NaPi pH 7 containing 0.5 mM PMSF, 0.5 mg/ml leupeptin, 0.7 mg/ml pepstatin A and 1 mg/ml aprotinin. The samples were frozen at −70° C. until further use. Protein concentration was determined according to the method described by Bradford (Bradford, 1976), using BSA as standard.

2-Dimensional Gel Electrophoresis

2-Dimensional gel electrophoresis was performed according to O'Farrell (O'Farrell and Goodman, 1976) using Bio-Rad's Mini-PROTEAN II Tube Cell with 1 mm gels. The first dimension gel monomer solution consisted of 9.2 M urea, 4% acrylamide, 20% Triton X-100, 2% Bio-Rad Biolyte 3/10 ampholyte 0.01% ammonium persulfate, 0.1% Temed. All but the last two reagents were mixed, and warmed with swirling at 45° C. until dissolved. The solution was briefly degassed, and the final two reagents were added under gentle swirling. The casting tubes were filled with monomer, which was allowed to polymerize at room temperature. The gels where then wrapped and stored at −20° C. In a sample preparation, 150 mg of Schwann cell protein was mixed with an equal volume of first dimension sample buffer consisting of 9.5 M urea 20% Triton X-100, 5% 2-EtSH, 2% Bio-Rad Biolyte 3/10. The protein was separated in the first dimension at 750 volts for 3.5 hours. For separation in the second dimension, a 10%, SDS-PAGE gel was cast as described (Weinstein et al., 1991). The gel from the separation in the first dimension was overlaid on the second gel and electrophoresis was performed at constant 70 mA. The gel was fixed for 30 min in 25% isopropanol and 10% acetic acid, and stained overnight in 10% acetic acid and 0.006% coomassie brilliant blue G250. Destaining was performed in 10% acetic acid. The gel was dried and photographed. Differentially expressed proteins from the ΔSCIP Schwann cells were excised and further analyzed by Mass Spectrometry.

Mass Spectrometry

The isolated protein spots were exposed to trypsin, separated on reversed phase HPLC and analyzed with a Finnigan electrospray mass spectrometer equipped with an iontrap. The resulting total ion current was then scanned for the presence of strong signals present for at least three scans. These peptide masses where then entered into a database (http://prospector.uscf.edu).

Cloning of OPA1

Degenerate primers were made which corresponded to the protein sequence deduced above. These were 5′GC(N)TC(N)GA(AG)CT(N)CT(N)GA(AG) 3′ and 5′TT(TC)AT(N)TC(N)TC(N)TC(N)GT(N)GG(N)3′. These primers were used to amplify a cDNA made from ΔSCIP Schwann cells, and a ˜1.1 kb product was generated. The PCR condition were 94° C. for 1 minute, followed by 40 cycles of 94° C. for 30 second, 54° C. for 3 minutes and 72° C. for 1 minute, followed by 72° C. for 5 minutes. The PCR product was the cloned using a TA cloning kit (Invitrogen), and sequenced. The cloned OPA1 fragment was then used to screen a mouse brain library (Stratagene) and a human fetal brain library (Clontech). All cloning was done as described (Weinstein et al., 1991), except the probe was generated by random priming instead of nick translation. All sequencing was carried out by automated sequencing on an ABI 310 automated sequencer.

Northern Blot Analysis

In brief, primary mouse Schwann cells were plated at 10⁶ cells/100 mm tissue culture dish in D¹⁰ and cultured for 48 hours in the absence of either GGF or forskolin. At time=0, the medium was replaced with fresh D¹⁰ and either GGF, FK506 or the FK506 vehicle (DMSO) were added to the cultures. Forty-eight hours later, total RNA was harvested. In the case of embryonic mouse neural tissue, embryos of the appropriate gestational age were delivered by cesarean section, and either the head (E9) or the brain (E 13) were harvested. In the case of adult mouse tissue, the animals were sacrificed by intracardiac delivery of a lethal dose of Avertine, the respective tissue dissected and RNA isolated. Total RNA was isolated from either tissue or cells as described (Chomczynski and Sacchi, 1987). 20 micrograms of total RNA was loaded into a 1% agarose/formaldehyde gel, as described (Weinstein et al., 1991), and electrophoresed at 100 V until the dye front moved 2/3 of the way down the length of the gel. The gel was rinsed in 2×SSC to remove excess formaldehyde, and the RNA transferred overnight to a Nytran nylon membrane, all as described (Weinstein et al., 1991). The filter was rinsed in 2×SSC, the RNA uv crosslinked, and the membrane was prehybridized for a minimum of 4 hours at 42° C. 25 ng of OPA1 probe was random prime labeled to a specific activity ³ 10⁹ CPM/mg of DNA, denatured, and hybridized to then membrane overnight at 42° C. The membrane was washed at 65° C. three times in 2×SSC/1% SDS and twice in 0.2×SSC/0.5% SDS, air dried and autoradiographed.

Transfection of OPA1 Into Wild-Type Schwann Cells

Partial mouse OPA1 cDNA, from an internal ATG through the stop codon, with the associated polyA site was cloned in frame behind the CMV promoter into the pCGS plasmid, and subsequently transfected into wild-type mouse Schwann cells, by calcium phosphate. A neomycin drug resistance gene on a separate plasmid (pRSVneo) was co-transfected, all as previously described (Weinstein et al., 1-991). The cells were grown in the neomycin analog G418, resistant clones were picked and expanded, and expression of OPA1 was determined by RTPCR (Weinstein et al., 1995). These cells were then used to make monolayers for assays of neurite outgrowth, as described above.

Results

Identification of SCIP-Expressed Proteins.

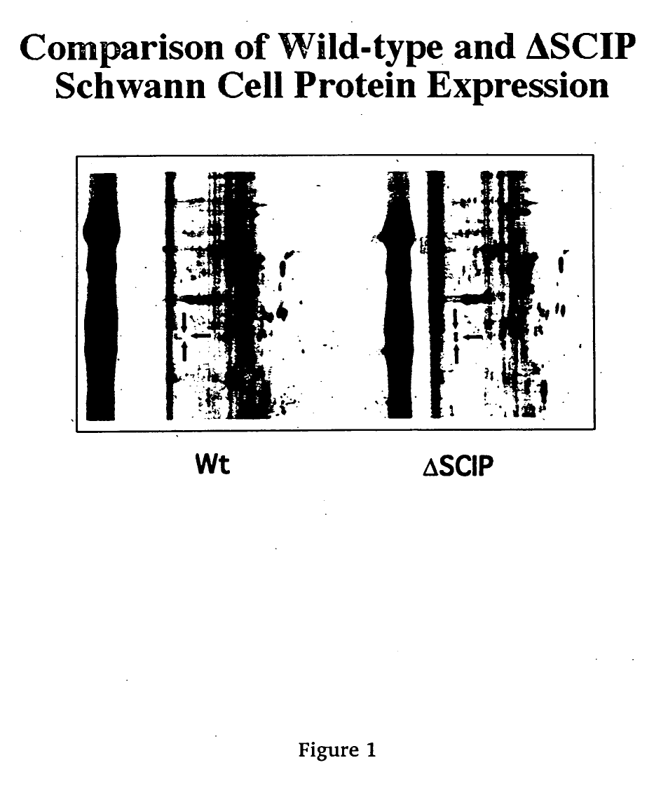

It has been previously demonstrated that mice expressing an NH₂ terminal truncation of the POU transcription factor ΔSCIP regenerate their peripheral nervous system (PNS) at an enhanced rate, and to a greater extent than wild-type animals (Gondré et al., 1998). In addition, the ability to support axon outgrowth was recapitulated in vitro, on monolayers of Schwann cells isolated from the ΔSCIP animals, and the activity was protein-associated (Gondré et al., 1998). In order to identify the protein(s) associated with this activity, a series of two-dimensional gel electrophoresis experiments was undertaken. As can be seen in FIG. 1, there are spots present in the ΔSCIP Schwann cell extract that are not present in extracts from the wild-type Schwann cells. Seven of the ΔSCIP-unique spots were excised from the gel and subjected to trypsinization, followed by mass spectroscopy. The peptide masses were compared to known masses in a protein data base (http://prospector.uscf.edu). One of the peptide sequences was suggestive of having a potential role in axon outgrowth, based on a limited homology to a recently described protein, termed neurocrescin (Nishimune et al., 1997).

Generation of an hOPA1 Partial cDNA

Degenerate PCR primers were synthesized corresponding to the peptide sequences identified by mass spectroscopy. These primers were used to amplify a cDNA made from cultured SCIP Schwann cells. The PCR product was cloned into the TA cloning vector, sequenced, amplified, and used to probe both mouse and human fetal brain cDNA libraries. Clones were picked, and taken through three rounds of purification. The resulting clones were then isolated, the cDNA inserts excised and sequenced. The primary sequence of the human clone is shown in FIG. 2B. Blast search of this sequence demonstrates identity with an region of human chromosome 17 (clone HCIT, Genbank accession # AC004148).

Expression Pattern of OPA1

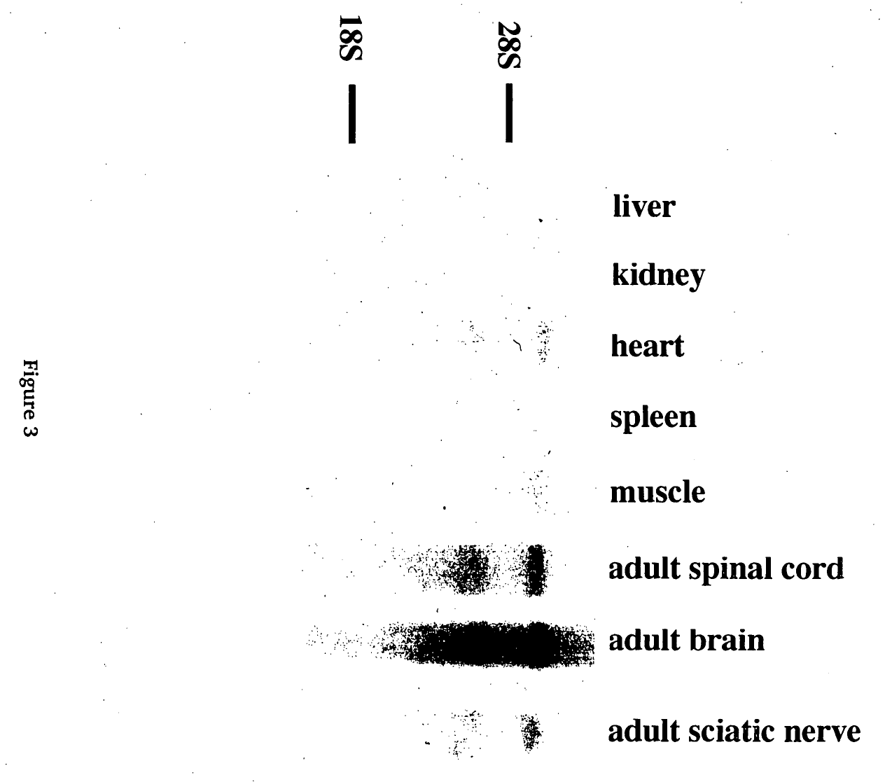

In order to determine the specificity of expression of the OPA1 clone, its expression in both adult and developing tissues was determined. A survey of a number of adult tissues demonstrates that OPA1 expression is restricted to neural tissues, as well as highly innervated tissues. OPA1 is expressed in adult mouse spinal cord and brain, as well as at lower levels in the adult sciatic nerve. There is modest expression in heart and skeletal muscle, both richly innervated tissues. There is no expression detected in liver kidney or spleen (FIG. 3).

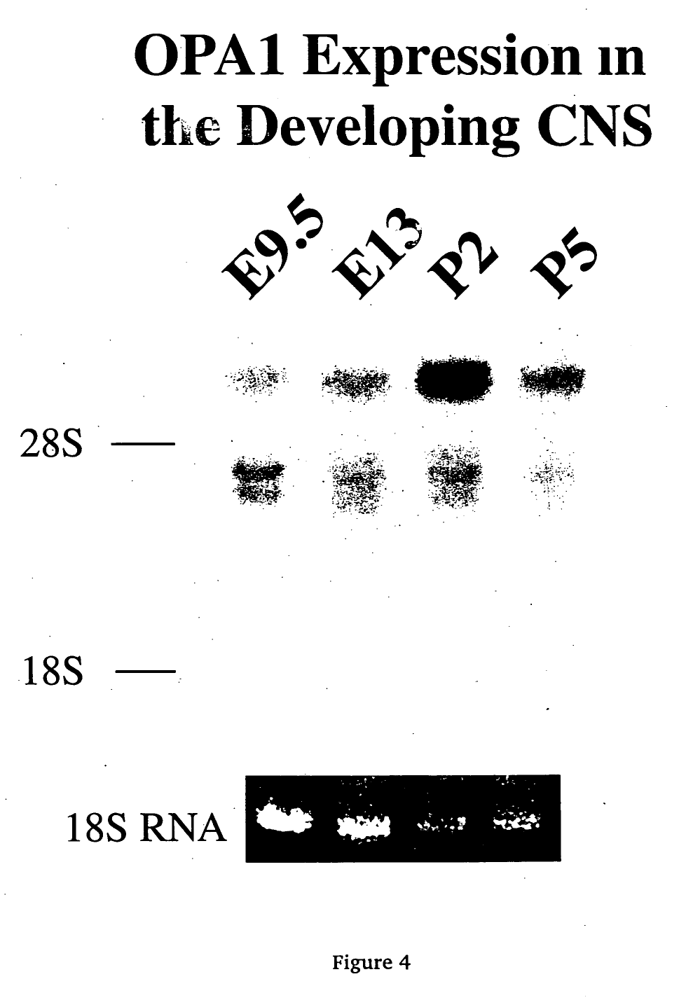

An analysis of OPA1 in the developing CNS shows a regulated pattern of expression, with three distinct bands that change in intensity over the course of development (FIG. 4). As early as embryonic day 9.5, the primary bands are a lower molecular weight doublet, and a less intense higher molecular weight band. By embryonic day 13, the lower bands appear as a smear of less intensity than at embryonic day 9.5, and the largest band has gained in intensity. At the second postnatal day, the highest band is clearly the most prominent, and the middle band is clearly seen, while the lowest band is indistinct. Just three days later, at postnatal day 5, the upper band is much less intense than at postnatal day 2, and the lower bands are a faint smear. The pattern of OPA1 expression at postnatal day 5 is very similar to that in the adult (compare to FIG. 3). Whether these three bands represent alternatively spliced variants of OPA1, or whether they represent three, related genes, is still unclear. However, the changes in expression of OPA1 correlate with major morphogenetic changes that occur over the course of development. These include neurogenesis at E9.5, neurogenesis and neuronal migration at E13, axonal outgrowth at P2, and the establishment and maintenance of neuronal homeostasis between P5 and adulthood.

Expression of OPA1 in Wild-type vs. ΔSCIP Schwann Cells

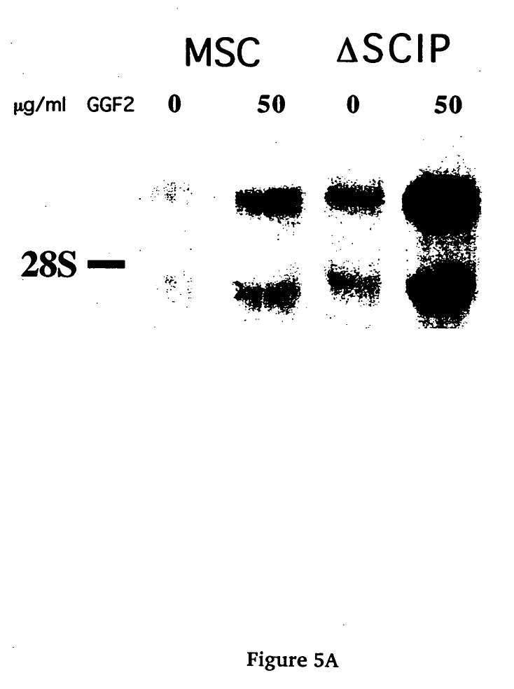

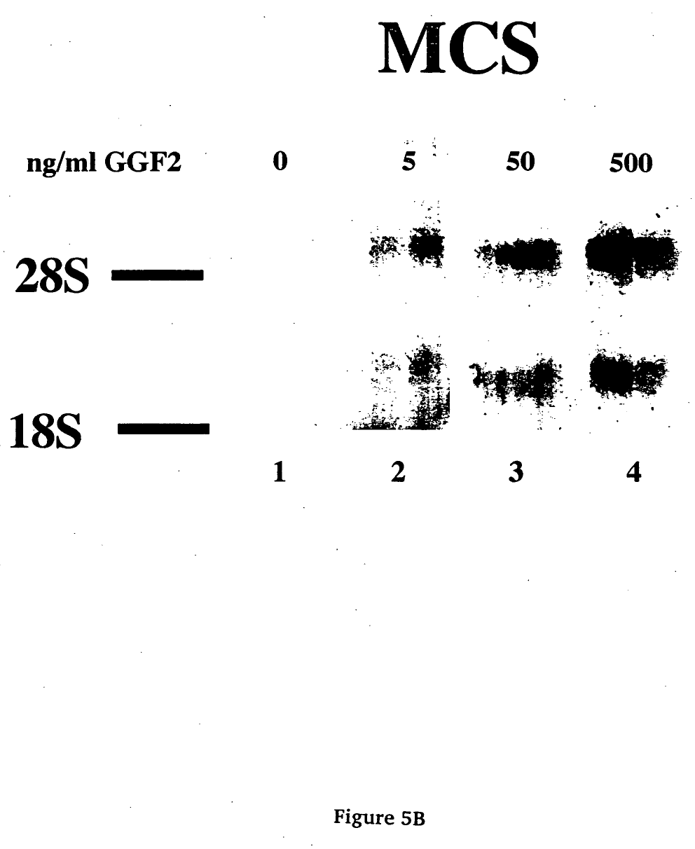

It was reasoned that because OPA1 protein was identified in a differential screen of wild-type Schwann cells vs. ΔSCIP Schwann cells, one might be able to detect differences in mRNA expression between the two cell types, and that such differences in expression might be further unveiled when the cells were exposed to glial growth factor (GGF). Moreover, the biology of PNS regeneration has been extensively studied, while the molecular mechanisms underlying that biology have remained somewhat obscure. However, it is known that Schwann cells are required for axonal regeneration, and that the regrowing axons express significant levels of GGF on their surface (as reviewed in Weinstein, 1999). Therefore, either wild-type or ΔSCIP Schwann cells were cultured either in D¹⁰, or in D¹⁰ supplemented with GGF. As can be seen in FIG. 5a, ΔSCIP cells constitutively express OPA1 mRNA, which is massively induced with 50 ng/ml of GGF. In contrast, the wild-type Schwann cells express no OPA1 at baseline, and it is induced to the baseline ΔSCIP level with the addition of 50 ng/ml of GGF. Notably, the upper and middle bands are expressed at approximately equivalent levels, while the lowest band is absent. A dose response curve of GGF on wild-type Schwann cells shows that expression of OPA1 plateaus at 50 ng/ml (FIG. 5b). In contrast, addition of greater than 50 ng/ml of GGF to ΔSCIP Schwann cells inhibits OPA1 expression (data not shown).

The Neuroimmunophilin FK506 Upregulates GGF Expression

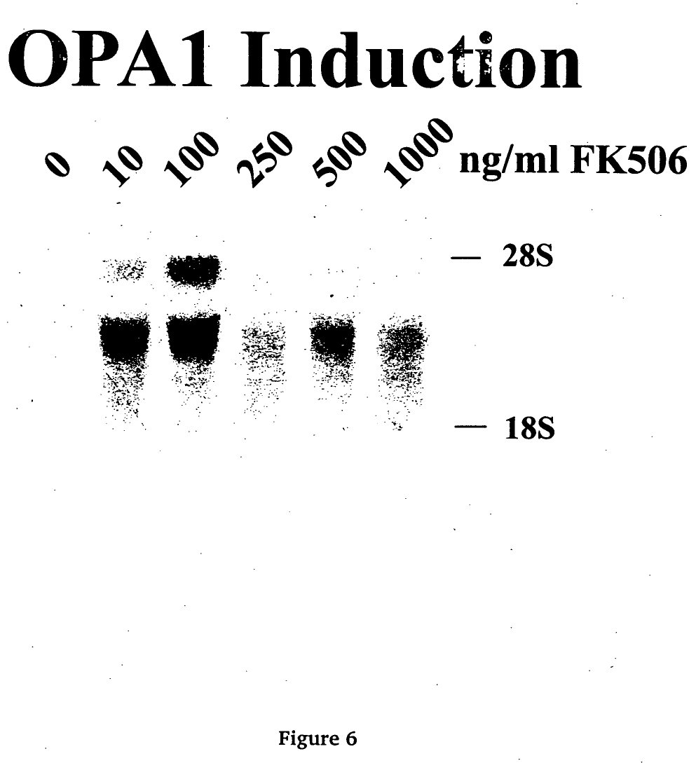

It has been recently appreciated that the immunosuppressive agent FK506 also stimulates nerve regeneration (Fansa et al., 1999; Gold, 1997; Gold et al., 1997; Hamilton and Steiner, 1998; Steiner et al., 1997). However, while these workers in this area have identified a number of potential FK506 targets in nerve stimulating nerve regeneration, including fkbp-12 and fkbp-52 (Gold et al., 1999; Gold et al., 1997; Steiner et al., 1997), these data are open to a wide range of interpretation, and neither protein has been directly tied to promoting axonal outgrowth. It was decided to test if the neuroimmunophilin FK506 is able to modulate OPA1 expression in cultured Schwann cells. A dose curve of the drug was added, dissolved in DMSO, to primary mouse Schwann cells, which were assayed for OPA1 induction after forty-eight hours. Interestingly, there is a bell-shaped induction of OPA1 by FK506, with strong induction at 10 nM and maximal induction at 100 nM (FIG. 6). However, above this dose, expression of OPA1 precipitously falls. Importantly, the induction of OPA1 with FK506 corresponds to the reported dosages at which FK506 has a therapeutic effect on nerve regeneration in animal models (Gold, 1999).

Neuronal Outgrowth on OPA1 Expressing Cells

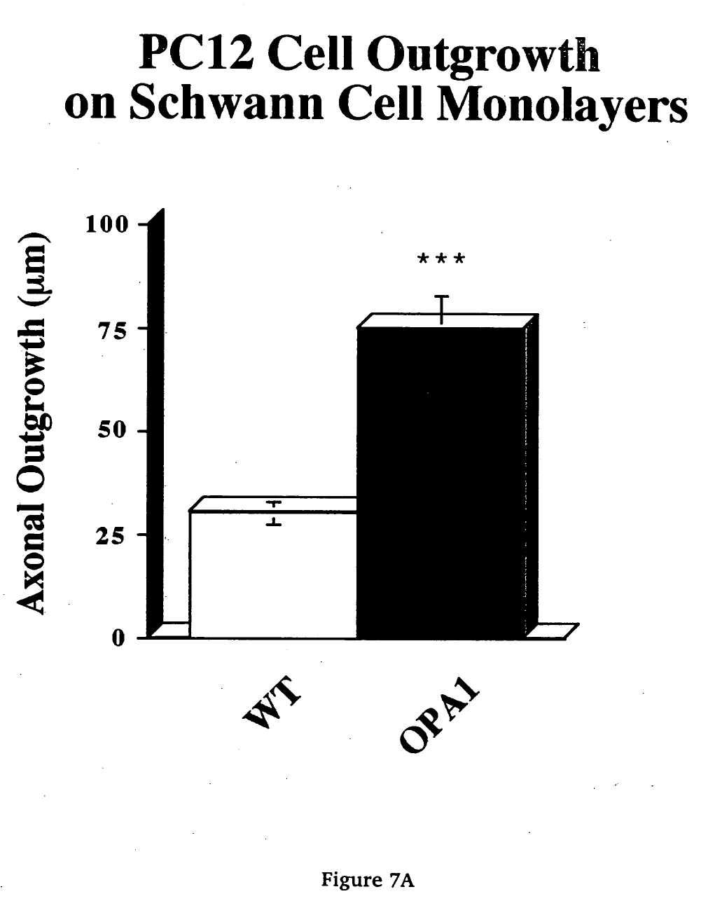

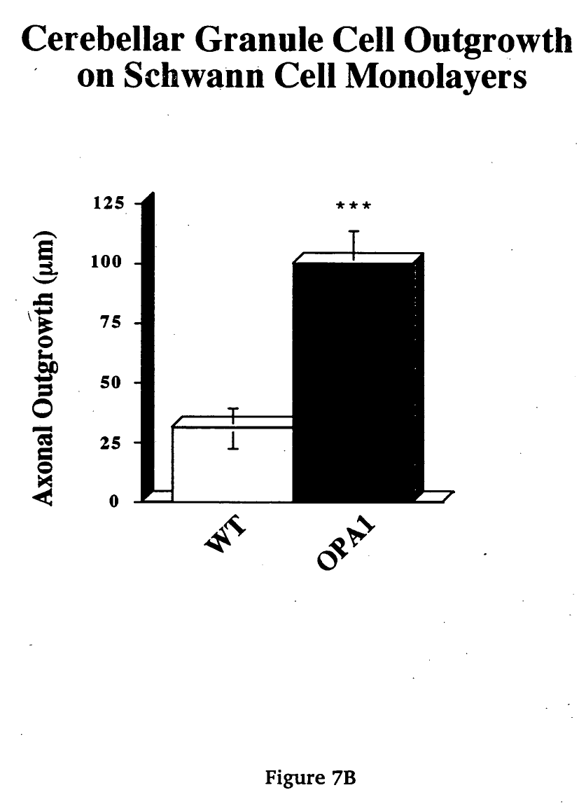

In order to determine if cells expressing OPA1 could recapitulate the enhanced regeneration shown by the ΔSCIP animals in vivo, a variety of neuronal types were cultured on OPA1 expressing cells. Enhanced outgrowth on of DRG neurons on ΔSCIP Schwann cells has been previously shown. Here, it was asked whether a PNS-like cell line, PC12, as well as central neurons could be maintained, and elaborate axons in co-culture with OPA1 expressing cells. PC12 cells normally grow as small, rounded cells unless they are exposed to differentiating agents, such as NGF (Tischler and Greene, 1978). Non-differentiated PC12 cells were plated on OPA1 expressing Schwann cells or on wild-type Schwann cells and then cultured for 24 hours. The cultures were fixed, and stained with TuJ1, a monoclonal antibody that recognizes a neuron-specific tubulin isoform (Easter et al., 1993), and the extent of process outgrowth was evaluated using the MetaMorph morphometry program. This analysis shows that PC12 cells extend axons that approach 100 mm overnight (FIG. 7a). To determine whether the outgrowth was the result of a previously unappreciated expression of NGF, the experiments were repeated in the presence of a neutralizing anti-NGF antibody (Sigma). No differences in PC12 cell axonogenesis in the presence or absence of the blocking antibody could be determined (data not shown).

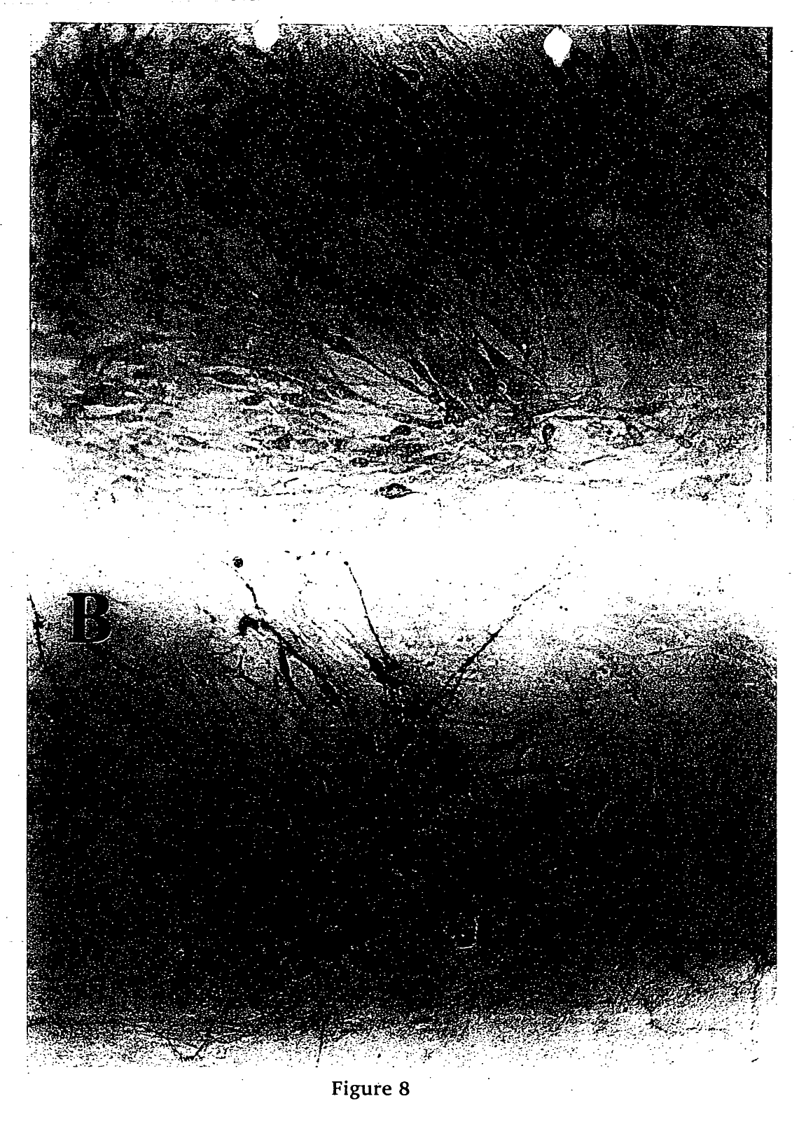

Following CNS injury, Schwann cells invade the CNS, where they often support and myelinate central axons for indefinite periods of time (as reviewed in Weinstein, 1999). To determine if Schwann cells expressing OPA1 were able to support CNS axons in vitro, cerebellar granule cells were cultured on monolayers of either wild-type or OPA1 expressing Schwann cells. There was a dramatic difference in granule cell survival in the presence of OPA1, as well as tremendous axonal outgrowth. Quantification of the outgrowth is shown in FIG. 7b, and an example of both cell survival and axonogenesis of the granule cell neurons can be seen in FIG. 8.

### REFERENCES

Aguayo, A. J., Peyronnard, J. M., and Bray, G. M. (1973). A quantitative ultrastructural study of regeneration from isolated proximal stumps of transected unmyelinated nerves. Journal of Neuropathology & Experimental Neurology 32, 256-70.
Albuquerque, M. L. C., Akiyama, S. K., and Schnaper, H. W. (1998). Basic Fibroblast Growth Factor Release by Human Coronary Artery Endothelial Cells Is Enhanced by Matrix Proteins, 17beta-Estradiol, and a PKC Signaling Pathway. Exp Cell Res 245, 163-169.
Baron-Van Evercooren, A., Gansmuller, A., Gumpel, M., Baumann, N., and Kleinman, H. K. (1986). Schwann cell differentiation in vitro: extracellular matrix deposition and interaction. Developmental Neuroscience 8, 182-96.
Bradford, M. M. (1976). A rapid and sensitive method for the quantitation of microgram quantities of protein utilizing the principle of protein-dye binding. Anal Biochem 72, 248-54.
Bray, G. M., Peyronnard, J. M., and Aguayo, A. J. (1972). Reactions of unmyelinated nerve fibers to injury. An ultrastructural study. Brain Research 42, 297-309.
Bunge, M. B., Williams, A. K., and Wood, P. M. (1982). Neuron-Schwann cell interaction in basal lamina formation. Developmental Biology 92, 449-60.
Carey, D. J., Eldridge, C. F., Cornbrooks, C. J., Timpl, R., and Bunge, R. P. (1983). Biosynthesis of type IV collagen by cultured rat Schwann cells. Journal of Cell Biology 97, 473-9.
Chen, M. S., Bermingham-McDonogh, O., Danehy, F. T., Jr., Nolan, C., Scherer, S. S., Lucas, J., Gwynne, D., and Marchionni, M. A. (1994). Expression of multiple neuregulin transcripts in postnatal rat brains. J Comp Neurol 349, 389-400.
Chomczynski, P., and Sacchi, N. (1987). Single-step method of RNA isolation by acid guanidinium thiocyanate-phenol-chloroform extraction. Analytical Biochemistry 162, 156-9.
Cornbrooks, C. J., Carey, D. J., McDonald, J. A., Timpl, R., and Bunge, R. P. (1983). In vivo and in vitro observations on laminin production by Schwann cells. Proceedings of the National Academy of Sciences of the United States of America 80, 3850-4.
Easter, S. S., Jr., Ross, L. S., and Frankfurter, A. (1993). Initial tract formation in the mouse brain. Journal of Neuroscience 13, 285-99.
Enver, M. K., and Hall, S. M. (1994). Are Schwann cells essential for axonal regeneration into muscle autografts? Neuropathol Appl Neurobiol 20, 587-98.
Fansa, H., Keilhoff, G., Altmann, S., Plogmeier, K., Wolf, G., and Schneider, W. (1999). The effect of the immunosuppressant FK 506 on peripheral nerve regeneration following nerve grafting. J Hand Surg [Br] 24, 38-42.
Feneley, M. R., Fawcett, J. W., and Keynes, R. J. (1991). The role of Schwann cells in the regeneration of peripheral nerve axons through muscle basal lamina grafts. Experimental Neurology 114, 275-85.
Friedman, H. C., Jelsma, T. N., Bray, G. M., and Aguayo, A. J. (1996). A distinct pattern of trophic factor expression in myelin-deficient nerves of Trembler mice: implications for trophic support by Schwann cells. J Neurosci 16, 5344-50.
Fujimoto, E., Mizoguchi, A., Hanada, K., Yajima, M., and Ide, C. (1997). Basic fibroblast growth factor promotes extension of regenerating axons of peripheral nerve. In vivo experiments using a Schwann cell basal lamina tube model. Journal of Neurocytology 26, 511-28.
Gold, B. G. (1997). FKS06 and the role of immunophilins in nerve regeneration. Mol Neurobiol 15, 285-306.
Gold, B. G. (1999). FK506 and the role of the immunophilin FKBP-52 in nerve regeneration. Drug Metab Rev 31, 649-63.
Gold, B. G., Densmore, V., Shou, W., Matzuk, M. M., and Gordon, H. S. (1999). Immunophilin FK506-binding protein 52 (not FK506-binding protein 12) mediates the neurotrophic action of FK506. J Pharmacol Exp Ther 289, 1202-10.
Gold, B. G., Zeleny-Pooley, M., Wang, M. S., Chaturvedi, P., and Armistead, D. M. (1997). A nonimmunosuppressant FKBP-12 ligand increases nerve regeneration. Exp Neurol 147, 269-78.
Gondré, M., Burrola, P., and Weinstein, D. E. (1998). Accelerated nerve regeneration mediated by Schwann cells expressing a mutant form of the POU protein SCIP. J Cell Biol 141, 493-501.
Griffin, J. W., George, E. B., and Chaudhry, V. (1996). Wallerian degeneration in peripheral nerve disease. Baillieres Clinical Neurology 5, 65-75.
Gulati, A. K. (1988). Evaluation of acellular and cellular nerve grafts in repair of rat peripheral nerve. Journal of Neurosurgery 68, 117-23.
Hall, S. M. (1986). Regeneration in cellular and acellular autografts in the peripheral nervous system. Neuropathology & Applied Neurobiology 12, 27-46.
Hall, S. M., and Kent, A. P. (1987). The response of regenerating peripheral neurites to a grafted optic nerve. Journal of Neurocytology 16, 317-31.
Hamilton, G. S., and Steiner, J. P. (1998). Immunophilins: beyond immunosuppression. J Med Chem 41, 5119-43.
Hammarberg, H., Risling, M., Hokfelt, T., Cullheim, S., and Piehl, F. (1998). Expression of insulin-like growth factors and corresponding binding proteins (IGFBP 1-6) in rat spinal cord and peripheral nerve after axonal injuries [In Process Citation]. J Comp Neurol 400, 57-72.
Ide, C., Tohyama, K., Yokota, R., Nitatori, T., and Onodera, S. (1983). Schwann cell basal lamina and nerve regeneration. Brain Research 288, 61-75.
Jenq, C. B., and Coggeshall, R. E. (1987). Sciatic nerve regeneration after autologous sural nerve transplantation in the rat. Brain Research 406, 52-61.
Kagami, S., Kondo, S., L#ster, K., Reutter, W., Urushihara, M., Kitamura, A., Kobayashi, S., and Kuroda, Y. (1998). Collagen Type I Modulates the Platelet-Derived Growth Factor (PDGF) Regulation of the Growth and Expression of beta1 Integrins by Rat Mesangial Cells. Biochem Biophys Res Commun 252, 728-732.
Le Beau, J. M., LaCorbiere, M., Powell, H. C., Ellisman, M. H., and Schubert, D. (1988). Extracellular fluid conditioned during peripheral nerve regeneration stimulates Schwann cell adhesion, migration and proliferation. Brain Research 459, 93-104.
Lindsay, R. M. (1994). Neurotrophic growth factors and neurodegenerative diseases: therapeutic potential of the neurotrophins and ciliary neurotrophic factor. [Review] [13 refs]. Neurobiology of Aging 15, 249-51.
Lisney, S. J. (1983). Changes in the somatotopic organization of the cat lumbar spinal cord following peripheral nerve transection and regeneration. Brain Research 259, 31-9.
Liuzzi, F. J., and Tedeschi, B. (1991). Peripheral nerve regeneration. Neurosurg Clin N Am 2, 31-42.
Luckenbill-Edds, L. (1997). Laminin and the mechanism of neuronal outgrowth. Brain Res Brain Res Rev 23, 1-27.
Mahanthappa, N. K., Anton, E. S., and Matthew, W. D. (1996). Glial Growth Factor 2, a Soluble Neuregulin, Directly Increases Schwann Cell Motility and Indirectly Promotes Neurite Outgrowth. J Neurosci 16, 4673-83.
Maisonpierre, P. C., Belluscio, L., Friedman, B., Alderson, R. F., Wiegand, S. J., Furth, M. E., Lindsay, R. M., and Yancopoulos, G. D. (1990). NT-3, BDNF, and NGF in the developing rat nervous system: parallel as well as reciprocal patterns of expression. Neuron 5, 501-9.
Marchionni, M. A., Goodearl, A. D., Chen, M. S., Bermingham-McDonogh, O., Kirk, C., Hendricks, M., Danehy, F., Misumi, D., Sudhalter, J., Kobayashi, K., and et al. (1993). Glial growth factors are alternatively spliced erbB2 ligands expressed in the nervous system [see comments]. Nature 362, 312-8.
Martin, G. R., Timpl, R., and Kuhn, K. (1988). Basement membrane proteins: molecular structure and function. Adv Protein Chem 39, 1-50.
McQuarrie, I. G. (1985). Effect of conditioning lesion on axonal sprout formation at nodes of Ranvier. Journal of Comparative Neurology 231, 239-49.
Meeker, M. L., and Farel, P. B. (1993). Coincidence of Schwann cell-derived basal lamina development and loss of regenerative specificity of spinal motoneurons. Journal of Comparative Neurology 329, 257-68.
Mehta, H., Orphe, C., Todd, M. S., Combrooks, C. J., and Carey, D. J. (1985). Synthesis by Schwann cells of basal lamina and membrane-associated heparan sulfate proteoglycans. Journal of Cell Biology 101, 660-6.
Meyer, D. B. (1995). Multiple essential functions of neuregulin in development. Nature 378, 390-394.
Monuki, E. S., Kuhn, R., Weinmaster, G., Trapp, B. D., and Lemke, G. (1990). Expression and activity of the POU transcription factor SCIP. Science 249, 1300-3.
Morgan, L., Jessen, K. R., and Mirsky, R. (1994). Negative regulation of the P0 gene in Schwann cells: suppression of P0 mRNA and protein induction in cultured Schwann cells by FGF2 and TGF beta 1, TGF beta 2 and TGF beta 3. Development 120, 1399-409.
Neuberger, T. J., and De Vries, G. H. (1993). Distribution of fibroblast growth factor in cultured dorsal root ganglion neurons and Schwann cells. I. Localization during maturation in vitro. Journal of Neurocytology 22, 436-48.
Nishimune, H., Uyeda, A., Nogawa, M., Fujimori, K., and Taguchi, T. (1997). Neurocrescin: a novel neurite-outgrowth factor secreted by muscle after denervation. Neuroreport 8, 3649-54.
O'Farrell, P. Z., and Goodman, H. M. (1976). Resolution of simian virus 40 proteins in whole cell extracts by two-dimensional electrophoresis: heterogeneity of the major capsid protein. Cell 9, 289-98.
Orr-Urtreger, A., Trakhtenbrot, L., Ben-Levy, R., Wen, D., Rechavi, G., Lonai, P., and Yarden, Y. (1993). Neural expression and chromosomal mapping of Neu differentiation factor to 8p12-p21. Proc Natl Acad Sci USA 90, 1867-71.
Porter, B. E., Weis, J., and Sanes, J. R. (1995). A motoneuron-selective stop signal in the synaptic protein S-laminin. Neuron 14, 549-59.
Rifkin, D. B., Moscatelli, D., Bizik, J., Quarto, N., Blei, F., Dennis, P., Flaumenhaft, R., and Mignatti, P. (1990). Growth factor control of extracellular proteolysis. Cell Differ Dev 32, 313-8.
Rosenbaum, C., Karyala, S., Marchionni, M. A., Kim, H. A., Krasnoselsky, A. L., Happel, B., Isaacs, I., Brackenbury, R., and Ratner, N. (1997). Schwann cells express NDF and SMDF/n-ARIA mRNAs, secrete neuregulin, and show constitutive activation of erbB3 receptors: evidence for a neuregulin autocrine loop. Exp Neurol 148, 604-15.
Roytta, M., and Salonen, V. (1988). Long-term endoneurial changes after nerve transection. Acta Neuropathologica 76, 35-45.
Salonen, V., Roytta, M., and Peltonen, J. (1987). The effects of nerve transection on the endoneurial collagen fibril sheaths. Acta Neuropathologica 74, 13-21.
Scaravilli, F., Love, S., and Myers, R. (1986). X-irradiation impairs regeneration of peripheral nerve across a gap. Journal of Neurocytology 15, 439-49.
Scherer, S. S., Wang, D. Y., Kuhn, R., Lemke, G., Wrabetz, L., and Kamholz, J. (1994). Axons regulate Schwann cell expression of the POU transcription factor SCIP. Journal of Neuroscience 14, 1930-42.
Sondell, M., Lundborg, G., and Kanje, M. (1998). Regeneration of the rat sciatic nerve into allografts made acellular through chemical extraction. Brain Research 795, 44-54.
Steiner, J. P., Hamilton, G. S., Ross, D. T., Valentine, H. L., Guo, H., Connolly, M. A., Liang, S., Ramsey, C., Li, J. H., Huang, W., Howorth, P., Soni, R., Fuller, M., Sauer, H., Nowotnik, A. C., and Suzdak, P. D. (1997). Neurotrophic immunophilin ligands stimulate structural and functional recovery in neurodegenerative animal models. Proc Natl Acad Sci USA 94, 2019-24.
Tischler, A. S., and Greene, L. A. (1978). Morphologic and cytochemical properties of a clonal line of rat adrenal pheochromocytoma cells which respond to nerve growth factor. Laboratory Investigation 39, 77-89.
Tohyama, K., and Ide, C. (1984). The localization of laminin and fibronectin on the Schwann cell basal lamina. Archivum Histologicum Japonicum-Nippon Soshikigaku Kiroku 47, 519-32.
Watson, D. F., Glass, J. D., and Griffin, J. W. (1993). Redistribution of cytoskeletal proteins in mammalian axons disconnected from their cell bodies. Journal of Neuroscience 13, 4354-60.
Webster, D. F. (1984). Development of peripheral nerve fibers. In In Peripheral Neuropathy, P. K. T. P. J. Dyck, E H. Lambert, R. Bunge, ed. (Philadelphia: W. B. Saunders).
Weinberg, H. J., and Spencer, P. S. (1978). The fate of Schwann cells isolated from axonal contact. Journal of Neurocytology 7, 555-69.
Weinstein, D. E. (1997). The Culture of Primary Astrocytes. In Current Protocols in Neuroscience, E. Crawley, C. Gerfen, R. McKay, M. Rogawski, D. Sibley and P. Skolnick, eds. (New York: Wiley and Son).
Weinstein, D. E. (1999). The Role of Schwann cells in Neural Regeneration. The Neuroscientist 5, 208-216.
Weinstein, D. E., Burrola, P. G., and Lemke, G. (1995). Premature Schwann cell differentiation and hypermyelination in mice expressing a targeted antagonist of the POU transcription factor SCIP. Molecular & Cellular Neurosciences 6, 212-29.
Weinstein, D. E., Shelanski, M. L., and Liem, R. K. (1990). C17, a retrovirally immortalized neuronal cell line, inhibits the proliferation of astrocytes and astrocytoma cells by a contact-mediated mechanism. Glia 3, 130-9.
Weinstein, D. E., Shelanski, M. L., and Liem, R. K. (1991). Suppression by antisense mRNA demonstrates a requirement for the glial fibrillary acidic protein in the formation of stable astrocytic processes in response to neurons. Journal of Cell Biology 112, 1205-13.
Wu, R., and Weinstein, D. E. (1999). Isolation and Purification of Primary Schwann Cells, Volume 1, J. N. Crawley, C. R. Gerfen, R. McKay, M. A. Rogawski, D. R. Sibley and P. Skolnick, eds. (New York: John Wiley & Sons).
Yurchenco, P. D., and Schittny, J. C. (1990). Molecular architecture of basement membranes. Faseb J 4, 1577-90.
Zhang, Y., Campbell, G., Anderson, P. N., Martini, R., Schachner, M., and Lieberman, A. R. (1995). Molecular basis of interactions between regenerating adult rat thalamic axons and Schwann cells in peripheral nerve grafts I. Neural cell adhesion molecules. Journal of Comparative Neurology 361, 193-209.

All publications mentioned herein, whether referring to an issued patent, patent application, published article or otherwise, are hereby incorporated by reference in their entirety. While the foregoing invention has been described in some detail for purposes of clarity and understanding, it will be appreciated by one skilled in the art from a reading of the disclosure that various changes in form and detail can be made without departing from the true scope of the invention in the appended claims.

1. An isolated nucleic acid encoding Opa1.

2. The isolated nucleic acid of claim 1 comprising the nucleotide sequence of nucleotides 880-1680.

3. An isolated nucleic acid of claim 1 comprising the nucleotide sequence of nucleotides 880-1680 of FIG. 2B.

4. The isolated nucleic acid of claim 1 having one or more mutations.

5. The nucleic acid of claim 4, wherein the mutations are selected from the group consisting of a point, insertion rearrangement or deletion mutation.

6. (canceled)

7. (canceled)

8. A purified Opa1 protein.

9. A purified Opa1 protein encoded by the nucleic acid of claim 1.

10. The purified Opa1 protein of claim 9, wherein the nucleic acid comprises the nucleotide sequence of nucleotides 880-1680 of FIG. 2A.

11. The purified Opa1 protein of claim 9, wherein the nucleic acid comprises the nucleotide sequence of nucleotides 880-1680 of FIG. 2B.

12. (canceled)

13. (canceled)

14. A vector comprising the nucleic acid of claim 1.

15. The vector of claim 14, wherein the nucleic acid comprises the nucleotide sequence of nucleotides 880-1680 of FIG. 2A.

16. The vector of claim 14, wherein the nucleic acid comprises the nucleotide sequence of nucleotides 880-1680 of FIG. 2B.

17. The vector of claim 14, wherein the nucleic acid hybridizes under high stringency conditions to a nucleic acid that is complementary to the nucleotide sequence of FIG. 2A or a contiguous fragment thereof.

18. The vector of claim 14, wherein the nucleic acid hybridizes under high stringency conditions to a nucleic acid that is complementary to the nucleotide sequence of FIG. 2B or a contiguous fragment thereof.

19. A host cell comprising the vector of claim 14.

20. The host cell of claim 19, wherein the nucleic acid comprises the nucleotide sequence of nucleotides 880-1680 of FIG. 2A.

21. The host cell of claim 19, wherein the nucleic acid comprises the nucleotide sequence of nucleotides 880-1680 of FIG. 2B.

22. The host of claim 19, wherein the nucleic acid hybridizes under high stringency conditions to a nucleic acid that is complementary to the nucleotide sequence of FIG. 2A or a contiguous fragment thereof.

23. The host of claim 19, wherein the nucleic acid hybridizes under high stringency conditions to a nucleic acid that is complementary to the nucleotide sequence of FIG. 2B or a contiguous fragment thereof.

24. (canceled)

25. (canceled)

26. (canceled)

27. (canceled)

28. A glial cell transduced with a vector, said vector comprising and expressing a nucleic acid encoding Opa1 protein, wherein the transduced cell provides biologically active Opa1 protein.

29. (canceled)

30. (canceled)

31. (canceled)

32. (canceled)
